(19) United States
(12) Patent Application Publication
(10) Pub. No.: US 2024/0082537 A1
(43) Pub. Date: Mar. 14, 2024

(54) CATHETER COVER FOR URINARY CATHETER

(71) Applicant: ORLANDO HEALTH, INC., Orlando, FL (US)

(72) Inventors: Miclat Minerva, Orlando, FL (US); Jack Stubbs, Orlando, FL (US)

(73) Assignee: ORLANDO HEALTH, INC., Orlando, FL (US)

(21) Appl. No.: 17/940,255

(22) Filed: Sep. 8, 2022

(51) Int. Cl. A61M 25/00

(52) U.S. Cl. CPC A61M 25/0017

(57) ABSTRACT

A cover for a urinary catheter may include a cover sheath, the cover sheath including a longitudinal slit extending from a first end of the sheath to a second end of the sheath. The catheter cover may also include and a spring mechanically coupled to the cover sheath and configured to retain the slit in a closed state, where the cover sheath substantially surrounds an outer diameter surface of the urinary catheter when the cover sheath is in the closed state.

### BACKGROUND

Urinary catheters of the type shown in FIG. 1 have been used successfully for many years. However, such catheters do have certain shortcomings. For example, common Foley catheters are indwelling catheters that remain in the body for days at a time, if not longer. These catheters need cleaning regularly (e.g., twice a day) via manual wiping via manual wiping with chlorhexidine wipes once a day and as needed. While such cleaning substantially reduces the chances of infection, it can be difficult and uncomfortable to clean areas very near to the body (e.g., near the patient's urethra). Further, since the catheter may slide slightly relative to the patient, the portions of the catheter immediately adjacent to the patient and/or immediately inside the patient are often overlooked and/or improperly cleaned. The embodiments discussed herein address these issues.

### BRIEF DESCRIPTION OF THE DRAWINGS

The embodiments discussed herein may be better understood with reference to the following drawings and description. The components in the figures are not necessarily to scale. Moreover, in the figures, like-referenced numerals designate corresponding parts throughout the different views.

FIG. 1 is an illustration showing a prior art urinary catheter.

FIG. 2 is an illustration showing a catheter cover for a urinary catheter or other catheter in accordance with certain aspects of the present disclosure, where a distal portion of the device is shown with a portion removed such that the internal area of the catheter cover is visible.

FIG. 3 is an illustration showing a second end of the catheter cover from FIG. 2.

FIG. 4 is an illustration showing a second end of the catheter cover from FIG. 2 having a portion removed such that the internal portion of the catheter cover is visible

FIG. 5 is an illustration showing a female body engagement portion of the catheter cover from FIG. 2

FIG. 6 is an illustration showing an additional view of the body engagement portion of the catheter cover from FIG. 2

FIG. 7 is an illustration of a catheter cover having a male body engagement portion in accordance with certain aspects of the present disclosure

FIG. 8 is an illustration of a catheter cover lacking a body engagement portion, but adapted for being secured to a selected body engagement portion, in accordance with certain aspects of the present disclosure.

FIG. 9 is an illustration of a catheter cover having another embodiment of a body engagement portion in accordance with certain aspects of the present disclosure.

FIGS. 10-12 are illustrations showing three embodiments of catheter covers for surrounding a urinary catheter with three respective sealing features in accordance with certain aspects of the present disclosure.

### DETAILED DESCRIPTION

Various aspects are described below with reference to the drawings in which like elements generally are identified by like numerals. The relationship and functioning of the various elements of the aspects may be better understood by reference to the following detailed description. However, aspects are not limited to those illustrated in the drawings or explicitly described below. It also should be understood that the drawings are not necessarily to scale, and in certain instances, details may have been omitted that are not necessary for an understanding of aspects disclosed herein, such as conventional fabrication and assembly.

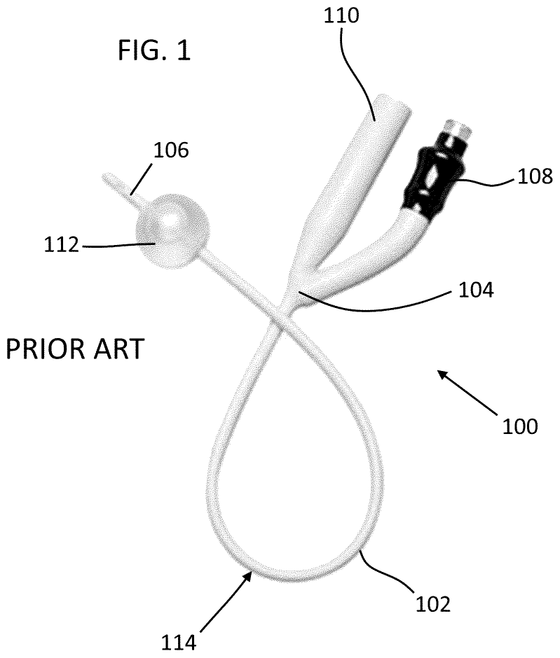

FIG. 1 is an illustration showing a urinary catheter 100 (e.g., a Foley catheter) common in the prior art. As shown, the urinary catheter 100 generally includes a catheter body 102 with a first end 104 and a second end 106. At the first

end 104, the urinary catheter 100 includes an inflation port 108 and a drainage port 110. The drainage port 110 provides access to a central lumen of the urinary catheter 100 and may be the opening by which urine is drained from the patient. The drainage port 110 may also operate as an interface for receiving a drainage tube, which may lead to a collection chamber. The inflation port 108 is typically in fluid communication with an inflation balloon 112 via a cavity between a

double-walled portion of the catheter body 102. When inflated, the balloon 112 secures the catheter in position within a patient's bladder.

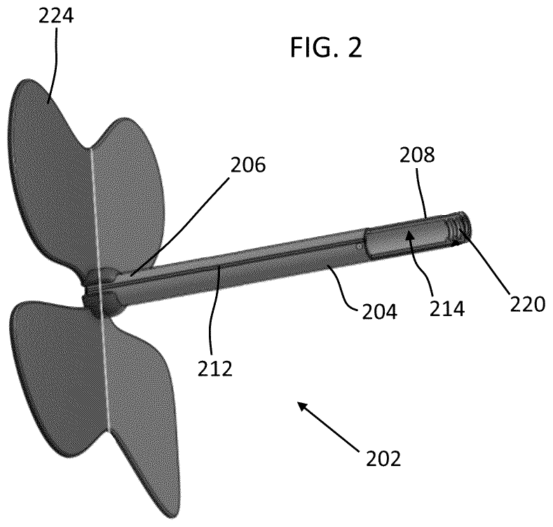

As discussed in the background above, urinary catheters of the type shown in FIG. 1 have been used successfully for many years but do have shortcomings. FIGS. 2-8 show various embodiments of catheter covers 202 that address these issues and provide an improved catheter environment. Notably, while the catheter covers 202 of this description primarily related to urinary catheters, similar catheter covers may be used for other types of catheters.

In particular, the catheter covers 202 may cover the portion of the urinary catheter 100 that is immediately adjacent to the patient to prevent soiling. Also, the catheter covers 202 may be removable (and perhaps disposable) such that they can be easily replaced, which may complement or potentially replace certain cleaning procedures. Further, the catheter covers 202 may engage the patient's body in a way such that sliding of the urinary catheter 100 relative to the patient is prevented or minimized, thereby enhancing the overall cleanliness of the urinary catheter 100.

Referring to FIGS. 2-4, the catheter cover 202 generally includes an elongated cover sheath 204 having a first end 206 and a second end 208. The cover sheath 204 may be made of any suitable material (e.g., silicone, polymer-based synthetics, etc.). In some embodiments, the cover sheath 204 may include, or be coated with, a microbial agent and/or a microbial-limiting material (such as silver, copper, etc.). Also, it is contemplated that at least a

portion of the cover sheath 204 may be transparent or translucent such that fluids within the urinary catheter remain visible.

The first end 206 of the catheter cover 202 is intended to be located adjacent to the patient's body, typically at the entrance to the patient's urethra. Optionally, the terminus of the first end 206 may contact the patient's body such that no (or little) space is located between the patient's body and the device's first end 206. As discussed in more detail below, the catheter cover 202 may include a body engagement portion 224 at its second end 208 configured for enhanced cleanliness

The catheter cover 202 may include a slit 212 along the length of the cover sheath 204. The slit 212 may be an elongated opening or other discontinuity that extends radially and generally interrupts the perimeter of the cover sheath 204. Advantageously, the slit 212 may provide the ability for the catheter cover 202 to be opened (e.g., via direct force from a medical professional's hands) and then wrapped around the outer diameter surface 114 of the urinary catheter 100 (e.g., once in place, the inner diameter surface 214 of the cover sheath 204 contacts the outer diameter surface of the urinary catheter 100).

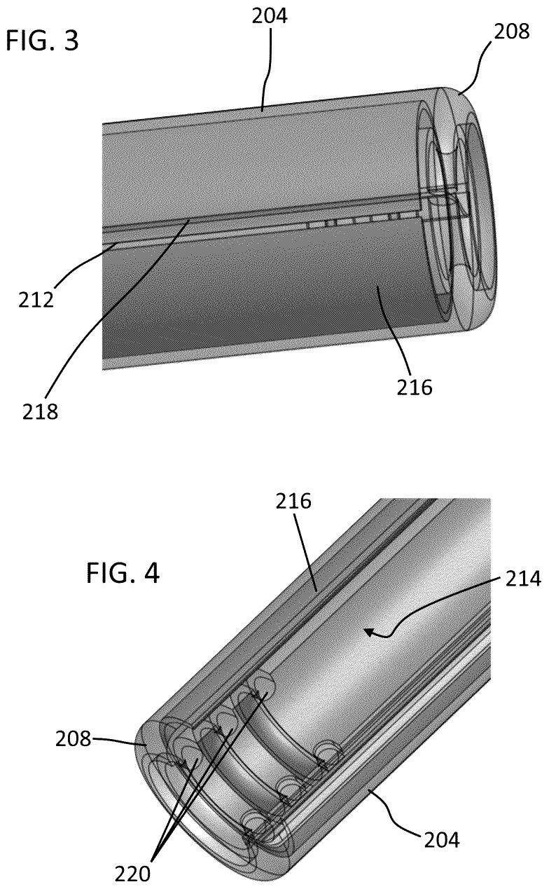

Referring to FIGS. 3-4, the catheter cover 202 may include a spring 216 that provides the cover sheath 204 with a tendency to wrap relatively tightly around the urinary catheter 100 when the catheter cover 202 is in its installed position. Advantageously, the spring 216 may provide adequate engagement (via contact pressure) between the catheter cover 202 and the underlying urinary catheter 100 such that slippage is prevented. In the depicted embodiment, the spring 216 is an elongated tube formed of a resilient material (e.g., a low-alloy steel or other metal, Nitinol, a resilient polyethylene or other synthetic material, etc.) and having a spring slit 218. Optionally, and as shown in FIGS. 3-4, the spring 216 may be inside, or surrounded by, the material forming the exposed surfaces (outer and inner) of the cover sheath 204, which may be advantageous for ensuring appropriate surface characteristics are not negatively affected by the spring (e.g., it may be desirable to cover the inner portion of the spring material with silicon for enhanced friction with the urinary catheter 100 and the outer portion of the spring to prevent the spring's metal from poking or otherwise contacting the patient).

Other spring types are also contemplated. For example, as an alternative (or in addition to) the spring 216, the catheter cover 202 may include a compression band (e.g., a rubber band) extending around the outer surface of the cover sheath 204, one or more magnets located at the slit 212, a tie extending around the circumference, or any other suitable device that imparts a tendency for the slit 212 to close. In certain embodiments, a separate spring may not be necessary, but rather the material aspects (including resiliency) of the cover sheath 204 itself may be sufficient for closing the device and providing adequate engagement with the urinary catheter 100.

Optionally, the catheter cover 202 may include one or more friction-enhancing devices for preventing a sliding motion of the cover sheath 204 relative to the urinary catheter 100. For example, referring to FIG. 3-4, three protrusions 220 are located at the second end 208 of the catheter cover 202 and extend from the inner diameter surface 214 of the cover sheath 204. Thus, when installed, these protrusions 220 may directly contact the outer diameter surface 114 of the urinary catheter 100, in some cases compressing against the urinary catheter 100 and/or compressing the outer-surface material of the urinary catheter 100 (thereby further enhancing the static friction between the devices). The spring 216 may radially overlap at least one of these protrusions 220 to enhance the contact pressure between the protrusions 220 and the urinary catheter 100, as shown.

While the protrusions 220 are located at the second end 208 of the device, they may be located in any other suitable location (i.e., anywhere along the length of the cover sheath's inner surface). For example, it is contemplated that substantially the entirety of the inner surface of the cover sheath 204 may include such protrusions for even more static friction. Also, it should be appreciated that more or fewer than three protrusions 220 may be included. The protrusions 220 may be unitary/integral with the cover sheath 204 such that they include the same material as that of the exposed surfaces of the cover sheath 204, but this is not required. By contrast, in other embodiments, similar protrusions may be separately assembled to the cover sheath 204 (e.g., O-rings may be placed in grooves within the inner-facing surface of the cover sheath 204).

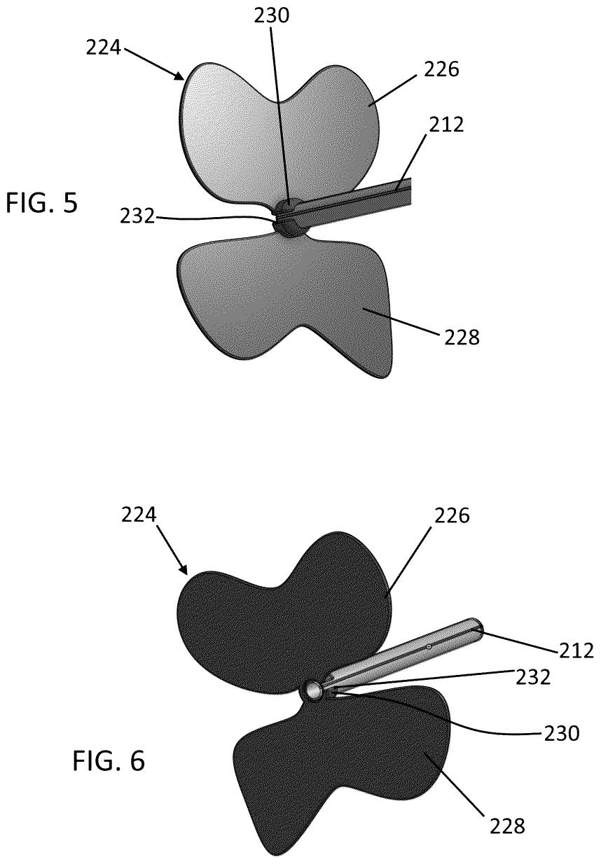

Optionally, the catheter cover 202 may include a body engagement portion, such as those shown in FIGS. 5-9. As discussed in more detail below, the body engagement portion (if included) may generally press against the patient and be adapted for the patient's particular anatomy. Advantageously, this device may prevent soiling of the urinary catheter near the urethral area and may substantially prevent or limit sliding action of the urinary catheter relative to the patient's urethra due to sufficient contact pressure with the patient in combination with high static friction relative to the urinary catheter.

Referring to FIGS. 5-6, the particular depicted body engagement portion includes a female contact device 224 generally sized and shaped for adapting to the anatomy surrounding a human female's urethral area. In particular, the device may include a set of panels, such as a first side 226 and a second side 228, which generally provides an organic profile and shape to fit to the anatomy of a female so that it is comfortable and covering.

One unique feature of the body engagement portion is the collar 230 where the body engagement portion is secured to the cover sheath 204. As shown, the collar 230 may substantially surround the cover sheath 204, and an inward radial force imparted by the collar 230 on the cover sheath 204 may generally prevent the body engagement portion from moving relative to the cover sheath 204. Optionally, the collar 230 may include a spring (much like the spring 216 discussed above), but this may not be required depending on the resilience and strength of the material used to form the collar 230. Further, the collar 230 may include a collar slit 232, which may radially align with the slit 212 of the cover sheath 204. Advantageously (or additionally), the collar 230 may be glued or otherwise adhered to the cover sheath 204.

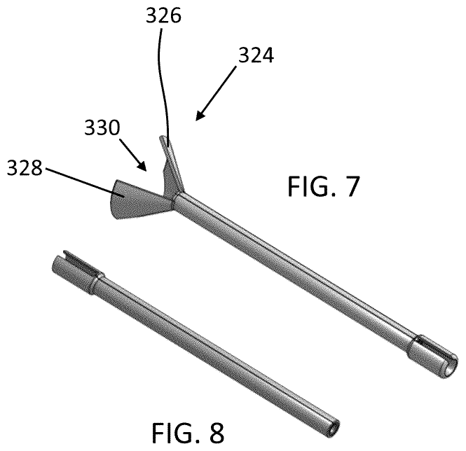

FIG. 7 shows another embodiment of a body engagement portion, which in this instance is a male contact device 324 configured for use on a male patient. As shown, the male contact device 324 includes a funnel-like structure having a first side 326 and a second side 328 that, when in use, generally cover the tip of the patient's penis. Advantageously, the funnel-like structure includes a slit or opening 330 that allows the device to open during installation around the urinary catheter 100. The male contact device 324 may be removeable such that the same catheter cover may be connectable to an appropriate body engagement portion (e.g., male, female, those of different sizes, or the like). E.g., a catheter cover lacking a body engagement portion, but adapted for being secured to a selected body engagement portion, is shown in FIG. 8.

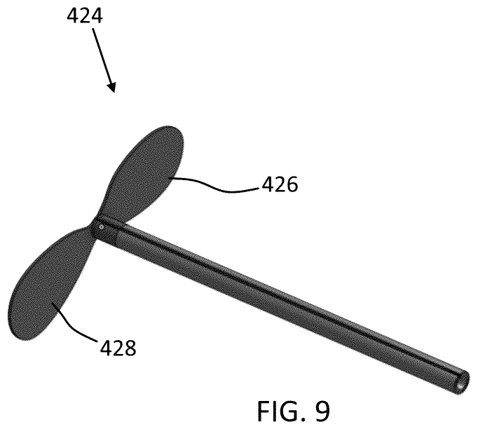

FIG. 9 shows another embodiment of a catheter cover having another embodiment of a body engagement portion 424. In this example, the body engagement portion 424 includes two wings or extensions 426 that extend radially away from a collar 430, which may be advantageous for manipulating body tissue of the female anatomy. Notably, when different body engagement portions are contemplated, these body engagement portions may be formed separately and may be attachable to the cover sheath (e.g., via the discussed collar). In other words, the body engagement portions may remain modular. This may be advantageous for allowing a medical professional to select an appropriate body-engagement portion that is appropriately sized and shaped for the patent while using a standardized cover sheath.

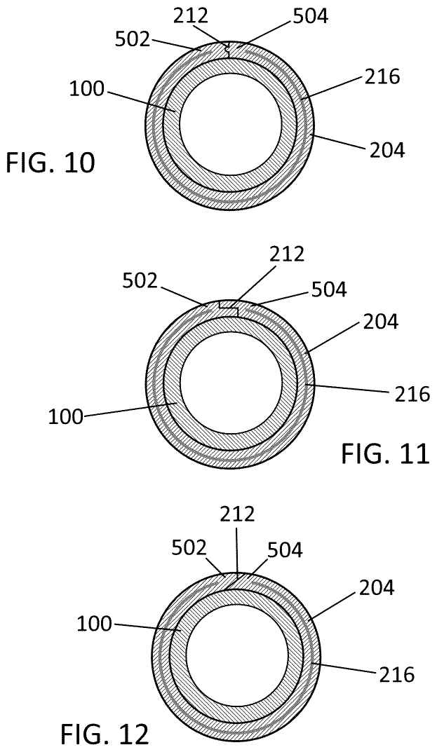

FIGS. 10-12 show three embodiments of the catheter cover 202 surrounding the urinary catheter 100, particularly to illustrate three of many possible ways to create a seal at the slit 212 of the cover sheath 204. For example, referring to FIG. 10, a first side 502 of the cover sheath 204 includes a depression that corresponds to a protrusion of a second side 504 of the cover sheath 204. In FIG. 11, the first side 502 of the cover sheath 204 includes a portion that extends beneath a corresponding portion of the second side 504. In FIG. 12, complementary angled surfaces of the first side 502 and the second side 504 engage one another. In each of these embodiments, the slit 212 is shaped such that there is no linear, radial path towards the urinary catheter 100 available for unwanted debris. Many other embodiments with different slit shapes are also contemplated.

While not shown, it is also contemplated that an optional locking device may be included at the slit 212 to prevent the cover sheath 204 from opening (although the spring 212 or other features discussed above may be sufficient on its own). For example, it is contemplated that a light adhesive may be applied to the seam. Other embodiments may include tape, a zipper, a hook, threads, wraps, or any other suitable locking device.

While various embodiments have been described, it will be apparent to those of ordinary skill in the art that many more embodiments and implementations are possible. Accordingly, the embodiments described herein are examples, not the only possible embodiments and implementations

Having described various aspects of the subject matter above, additional disclosure is provided below that may be consistent with the claims originally filed with this disclosure. In describing this additional subject matter, reference may be made to the previously described figures. Any of the following aspects may be combined, where compatible.

One general aspect includes a catheter cover for a urinary catheter, a cover sheath, the cover sheath including a longitudinal slit extending from a first end of the sheath to a second end of the sheath. The catheter cover also includes and a spring mechanically coupled to the cover sheath and configured to retain the slit in a closed state, where the cover sheath substantially surrounds an outer diameter surface of the urinary catheter when the cover sheath is in the closed state.

Implementations may include one or more of the following features. The spring may include an elongated tube having a spring slit that is coextensive with the slit of the cover sheath. The spring may substantially surround by a material forming the cover sheath. The catheter cover may include at least one friction protrusion located at the second end of the cover sheath, the at least one friction protrusion configured to contact the outer diameter surface of the urinary catheter when the cover sheath is in the closed state. The spring may radially overlap the at least one friction protrusion. The at least one friction protrusion may be integral with an inner surface of the cover sheath. The catheter cover may also include a body engagement portion extending radially outward from the cover sheath and located at the first end of the catheter cover. The body engagement portion may include a collar securing the body engagement portion to the cover sheath. The collar may include a collar slit that is coextensive with the slit of the cover sheath. The body engagement portion may be modular relative to the cover sheath.

Another general aspect may include a catheter cover for a urinary catheter, a cover sheath, the cover sheath including a longitudinal slit extending from a first end of the sheath to a second end of the sheath. The catheter cover also may include and a body engagement portion extending radially outward from the cover sheath and located at the first end of the catheter cover.

Implementations may include one or more of the following features. The catheter cover may include a spring mechanically coupled to the cover sheath, where the spring may include an elongated tube having a spring slit that is coextensive with the slit of the cover sheath. The catheter cover may include at least one friction protrusion located at the second end of the cover sheath, the at least one friction protrusion configured to contact an outer diameter surface of the urinary catheter when the cover sheath is in a closed state. The body engagement portion may include a collar securing the body engagement portion to the cover sheath, and where the collar may include a collar slit that is coextensive with the slit of the cover sheath. The body engagement portion may be modular relative to the cover sheath. The body engagement portion may be adapted for an anatomy of a female patient and may include a set of panels. The body engagement portion may be adapted for an anatomy of a male patient and may include a funnel-like structure.

Another general aspect includes a method that includes forming a urinary catheter consistent with any of the aspects discussed above.

We claim:

1. A catheter cover for a urinary catheter, comprising:
a cover sheath, the cover sheath including a longitudinal slit extending from a first end of the sheath to a second end of the sheath; and
a spring mechanically coupled to the cover sheath and configured to retain the slit in a closed state, wherein the cover sheath substantially surrounds an outer diameter surface of the urinary catheter when the cover sheath is in the closed state.

2. The catheter cover of claim 1, wherein the spring includes an elongated tube having a spring slit that is coextensive with the slit of the cover sheath.

3. The catheter cover of claim 2, wherein the spring is substantially surrounded by a material forming the cover sheath.

4. The catheter cover of claim 1, further comprising at least one friction protrusion located at the second end of the cover sheath, the at least one friction protrusion configured to contact the outer diameter surface of the urinary catheter when the cover sheath is in the closed state.

5. The catheter cover of claim 4, wherein the spring radially overlaps the at least one friction protrusion.

6. The catheter cover of claim 4, wherein the at least one friction protrusion is integral with an inner surface of the cover sheath.

7. The catheter cover of claim 1, further comprising a body engagement portion extending radially outward from the cover sheath and located at the first end of the catheter cover.

8. The catheter cover of claim 7, wherein the body engagement portion includes a collar securing the body engagement portion to the cover sheath.

9. The catheter cover of claim 8, wherein the collar includes a collar slit that is coextensive with the slit of the cover sheath.

10. The catheter cover of claim 7, wherein the body engagement portion is modular relative to the cover sheath.

11. A catheter cover for a urinary catheter, comprising:
a cover sheath, the cover sheath including a longitudinal slit extending from a first end of the sheath to a second end of the sheath; and
a body engagement portion extending radially outward from the cover sheath and located at the first end of the catheter cover.

12. The catheter cover of claim 11, further comprising a spring mechanically coupled to the cover sheath, wherein the spring includes an elongated tube having a spring slit that is coextensive with the slit of the cover sheath.

13. The catheter cover of claim 11, further comprising at least one friction protrusion located at the second end of the cover sheath, the at least one friction protrusion configured to contact an outer diameter surface of the urinary catheter when the cover sheath is in a closed state.

14. The catheter cover of claim 11, wherein the body engagement portion includes a collar securing the body engagement portion to the cover sheath, and wherein the collar includes a collar slit that is coextensive with the slit of the cover sheath.

15. The catheter cover of claim 11, wherein the body engagement portion is modular relative to the cover sheath.

16. The catheter cover of claim 11, wherein the body engagement portion is adapted for an anatomy of a female patient and includes a set of panels.

17. The catheter cover of claim 11, wherein the body engagement portion is adapted for an anatomy of a male patient and includes a funnel-like structure.

18. A method, comprising:
forming a catheter cover for a urinary catheter,
wherein the catheter cover includes a cover sheath, the cover sheath including a longitudinal slit extending from a first end of the sheath to a second end of the sheath, and
wherein the catheter cover includes a spring mechanically coupled to the cover sheath and configured to retain the slit in a closed state, wherein the cover sheath substantially surrounds an outer diameter surface of the urinary catheter when the cover sheath is in the closed state.

19. The method of claim 18, wherein the spring includes an elongated tube having a spring slit that is coextensive with the slit of the cover sheath.

20. The method of claim 18, wherein the spring includes a body engagement portion extending radially outward from the cover sheath and located at the first end of the catheter cover.
